(19) United States
(12) Patent Application Publication
(10) Pub. No.: US 2023/0139835 A1
(43) Pub. Date: May 4, 2023

(54) ALLULOSE IN CRYSTALLINE FORM

(71) Applicant: SAVANNA INGREDIENTS GMBH, Elsdorf (DE)

(72) Inventors: Vanessa BACHMANN, Mönchengladbach (DE); Stephan MOHR, Eushkirchen (DE); Birgit KAUFMANN, Köln (DE); Carolin Ellen KLEIN, Wesseling (DE); TIMO KOCH, Elsdorf (DE); Anna JUNKLEWITZ, Wuppertal (DE); Nathalie VEHLOW, Frechen (DE); Steffen BUTZ, Kreuzau (DE)

(21) Appl. No.: 17/798,548

(22) PCT Filed: Feb. 10, 2021
(86) PCT No.: PCT/EP2021/053255

§ 371 Date: Aug. 9, 2022

(30) Foreign Application Priority Data
Feb. 12, 2020 (EP) 20156808.6
Feb. 12, 2020 (EP) 20156862.3

(51) Int. Cl. C07H 3/02; C07H 1/06; A61K 47/26; A61K 8/60; A61Q 11/00; A23L 27/30; A23L 2/60; A23L 29/30; A23L 27/00; A21D 2/18; A23G 1/40; A23G 3/42; A23G 4/10; A23C 9/13

(52) U.S. Cl. CPC C07H 3/02; C07H 1/06; A61K 47/26; A61K 8/60; A61Q 11/00; A23L 27/33; A23L 2/60; A23L 29/30; A23L 27/88; A23L 27/84; A21D 2/181; A23G 1/40; A23G 3/42; A23G 4/10; A23C 9/1307; C07B 2200/13; A61K 2800/28; A61K 2800/92

(57) ABSTRACT

The invention relates to a crystalline allulose with a defined particle size distribution, to a process for the production thereof, as well as to the use thereof. The new allulose quality is characterized by the fact that it improves the shelf life of end products made therewith as well as their sensory and taste properties.

### FIELD OF THE INVENTION

The invention is in the field of food technology and relates to a new crystalline allulose characterized by a specific particle size distribution, to a process for the production thereof, as well as to the use thereof for the production of oral preparations.

### TECHNICAL BACKGROUND

Due to the growing interest of a large part of the population in “healthy eating” and “healthy living” in general, no-calorie or at least low-calorie sugars and sugar substitutes have attracted a great deal of interest in the food industry and in the scientific community.

Sugar substitutes include sweeteners and sugar alcohols. Sweeteners can be made naturally or synthetically. They are characterized by having no or negligible nutritional value, which makes them particularly interesting for people on a diet.

The body excretes these sweeteners either completely or mostly unchanged. They can be found in many low-calorie and diet products. Sugar substitutes are carbohydrates that only cause a slight increase in blood sugar and insulin levels. Their sweetening power is 40 to 70 percent the sweetening power of normal table sugar. The proportion of sugar substitutes in food and beverages has increased continuously in recent years. In the context of the present invention, the term “low-calorie” should be understood to mean a calorific value of not more than 10, preferably not more than 5, and in particular from 1 to 2 kcal.

The most important sugar substitutes industrially are acesulfame K and aspartame. These are synthetic substances that some studies have identified as carcinogenic. However, these hitherto unconfirmed findings strengthen the general trend towards “natural” sugar substitutes.

The most important natural sugar substitutes include stevia, erythritol, xylitol, sucralose, and sorbitol. The sweetening power of these substances compared to sucrose is between 0.5 and 600, and the number of calories is between 0 and 260. However, all these substances have disadvantages, for example:
Stevia has an intense, unpleasant, metallic taste of its own and is unsuitable for baking;
Erythritol and xylitol cannot be caramelized or browned (Maillard reaction);
Sucralose is unsuitable for people with fructose intolerance;
Many people, particularly diabetics, cannot tolerate sorbitol.

But there is also room for improvement in the ready-made products, starting with the improved shelf life of food in general or the lack of color stability of tomato ketchup in particular, the unsatisfactory solubility of many instant beverages, especially in the vending machine sector, and the sensory effects such as the lack of creaminess of ice cream, yogurt, or cream cheese, to name just a few exemplary deficiencies.

These deficiencies could be eliminated by the so-called “rare sugars” such as tagatose, cellobiose, or allulose (which is also synonymously referred to as psicose). These are simple sugars that do not have the sweetening power of table sugar but are not metabolized by the human body. They are also considered “natural” because they can be found in nature, albeit in small amounts. They are therefore particularly suitable for partial replacement in foods containing sugar.

Allulose (psicose) is a low-calorie sugar with a sweet taste similar to sugar. Allulose is one of many different sugars that occur in very small amounts in nature. Allulose was originally identified from wheat and has since been found in certain fruits such as jackfruit, figs, and raisins. Allulose is found naturally in small amounts in a variety of sweet foods like caramel sauce, maple syrup, and brown sugar. Allulose is absorbed by the body but not metabolized, making it nearly calorie-free.

### RELEVANT PRIOR ART

Various processes for the production of allulose are known from the prior art, for example, from documents WO 2018/087261 A1 (Pfeifer & Langen GmbH & Co. KG) or WO 2016/135358 A1 (Tate & Lyle Technology Limited).

WO 2018 20103 A1 (GIVAUDAN) claims a sweetener mixture of stevia and other sweeteners such as psicose. Psicose is not at the forefront but rather mogrosides.

WO 2014 186084 A1 (PEPSICO) claims a beverage composition which contains erythrol, psicose, and rebaudioside M as sweetener components.

### OBJECT OF THE INVENTION

The shelf life of food is described with the help of water activity or the so-called a_{w} value. This is a measure of the “available” or “active” water as opposed to just the water content. The importance of this variable is due to the fact that not only the pure water content is important for the shelf life of food but also the extent to which the water is bound by the substrate. Water activity affects the growth of microorganisms, the occurrence of chemical processes such as fat oxidation and non-enzymatic browning, the activity of enzymes, and the physical properties of the food. Water activity is defined as the ratio between the water vapor partial pressure in the food (p) and the saturation vapor pressure of pure water (p₀) at a given temperature:

a_{w}=p/P₀

Water activity is synonymous with equilibrium (relative) humidity (ERH), i.e. the relative humidity at which the food (again at the given temperature) is in equilibrium with the surrounding air, i.e. neither losing nor absorbing water. However, relative humidity is most often expressed in the auxiliary unit of percent, so equilibrium relative humidity is calculated as:

ERH=a_{w}*100

At the same time, the water activity (a_{w}) 0-1 corresponds to a relative humidity of 0-100%. In the simplest case, water activity is measured by placing a sample of the food in a hermetically sealed container and measuring the humidity in the container with a hygrometer. https://de.wikipedia.org/wiki/Aw-Wert-cite_note-Ternes_2008-4

A lowering of the water value—synonymous with the reduction of the available water—thus represents an ongoing challenge for the food industry to improve the shelf life of their products.

The object of the present invention was therefore to provide a non-caloric sugar substitute in a new form which extends the shelf life of foods and improves the taste and sensory properties of end preparations which can contain sugar or sugar substitutes.

### DESCRIPTION OF THE INVENTION

The subject matter of the present invention is allulose in crystalline form, characterized in that it has a particle size distribution determined by the X_{c(min)} method, to which one, two, or all three of the following selection rules apply:

| Dispersion pressure | Minimum d10 | Minimum d50 | Minimum d90 |
|---|---|---|---|
| [kPa] | [μm] | [μm] | [μm] |
| 5 | >70 | >140 | >230 |
| 20 | >50 | >120 | >220 |
| 460 | >10 | >40 | >130 |

and/or

it has a particle size distribution determined by the X_{Fe(max)} method, to which one, two, or all three of the following selection rules apply:

| Dispersion pressure | Minimum d10 | Minimum d50 | Minimum d90 |
|---|---|---|---|
| [kPa] | [μm] | [μm] | [μm] |
| 5 | >100 | >220 | >340 |
| 20 | >70 | >200 | >330 |
| 460 | >8 | >65 | >170 |

and/or

it has a particle size distribution determined by the Xₐᵣₑₐ method, to which one, two, or all three of the following selection rules apply:

| Dispersion pressure | Minimum d10 | Minimum d50 | Minimum d90 |
|---|---|---|---|
| [kPa] | [μm] | [μm] | [μm] |
| 5 | >85 | >185 | >265 |
| 20 | >65 | >180 | >260 |
| 460 | >10 | >50 | >120 |

Said preferred particle size distribution is determined by means of dynamic image analysis (Camsizer X2 from Retsch with X-Jet module), wherein a dispersion pressure of 5, 20, or 460 kPa was applied. The evaluation was carried out in three size models, namely:

X_{c(min)} Particle width which is determined from the narrowest of all measured chords x_{c};

X_{Fe(max)} Particle length which is determined from the longest of all measured Feret diameters x_{Fe};

Xₐᵣₑₐ Equivalent particle diameter which corresponds to the circle equivalent diameter.

The value “b/l d50” indicates the respective width/length ratio of the crystals at d50. It is particularly preferred if this value is between 0.6 and 1.0, in particular between 0.7 and 0.9, and optimally between 0.75 and 0.8.

In a preferred embodiment, the allulose according to the invention in crystalline form has a b/l d50 value, to which the following applies:

| | Dispersion pressure | |
|---|---|---|
| Parameter | [kPa] | X_{c(min)} |
| b/1 d50 | 5 | 0.52-1.0 |

and/or

| | | |
|---|---|---|
| b/1 d50 | 20 | 0.53-1.0 |

and/or

| | | |
|---|---|---|
| b/1 d50 | 460 | 0.60-1.0 |

and/or

| | Dispersion pressure | |
|---|---|---|
| Parameter | [kPa] | X_{Fe(max)} |
| b/1 d50 | 5 | 0.52-1.0 |

and/or

| | | |
|---|---|---|
| b/1 d50 | 20 | 0.55-1.0 |

and/or

| | | |
|---|---|---|
| b/1 d50 | 460 | 0.65-1.0 |

and/or:

| | Dispersion pressure | |
|---|---|---|
| Parameter | [kPa] | Xₐᵣₑₐ |
| b/l d50 | 5 | 0.52-1.0 |

and/or

| | | |
|---|---|---|
| b/l d50 | 20 | 0.55-1.0 |

and/or

| | | |
|---|---|---|
| b/l d50 | 460 | 0.65-1.0 |

Surprisingly, it was found that allulose, as a sweetener, can reduce the water content of ready-made products and thus significantly improve the shelf life thereof if they have a defined particle size distribution as described above. This was particularly surprising and not to be expected since a connection between the diameter and shape of crystal particles and the water value was not previously known. At the same time, this new crystalline allulose quality is able to positively influence the sensory and taste properties of oral preparations overall and of foods in particular in many ways, such as:

Improvement of mouthfeel;

Color intensification and stabilization, e.g. in orange lemonade;

Enhancement of flavor intensity, e.g. more intense fruitiness of the tomato in ketchup or the umami flavor in snack items;

Stabilization of food emulsions; or

Faster solubility of instant products just to name a few.

As previously mentioned, allulose (psicose) is a low-calorie sugar with a sweet taste similar to sugar. Allulose is one of many different sugars that occur in very small amounts in nature. Allulose was first identified from wheat and has since been found in certain fruits such as jackfruit, figs, and raisins. Allulose is found naturally in small amounts in a variety of sweet foods like caramel sauce, maple syrup, and brown sugar. Allulose is absorbed by the body but not metabolized, making it nearly calorie-free.

The production of allulose or psicose from fructose has been known since 1995, as the selection of the following publications shows:

In Journal of Fermentation Bioengineering, 80(1), 1995, pgs. 101-103, H. Itoh, et al. discloses the production of D-psicose from D-fructose by means of immobilized D-tagatose 3-epimerase.

In Organic Process Research Development 2012, 16, pgs. 323-330, N. Wagner, et al. refers to practical aspects of the integrated operation of biotransformation and simulated moving bed (SMB) separation for fine chemical synthesis. D-psicose is produced from D-fructose using epimerization catalyzed with D-tagatose epimerase.

In Chemical Engineering Science 137 (2015) pgs. 423-435, N. Wagner, et al. refers to the model-based cost optimization of an integrated process for the enzymatic production of psicose at elevated temperatures.

In Angewandte Chemie [Applied Chemistry] Int. Ed. Engl. 2015, 54(14), pgs. 4182-6, N. Wagner, et al. discloses a separation-integrated cascade reaction to overcome thermodynamic limitations in rare sugar synthesis.

In Biotechnology Bioengineering 2016, 113(2), pgs. 349-58, Bosshart, et al. refers to the production of the rare sugars D-psicose and L-tagatose by two artificially produced D-tagatose epimerases.

In Journal of Chromatography A 2015, 1398, pgs. 47-56, N. Wagner, et al. discloses a process for the economical production of D-psicose by means of simulated moving bed chromatography.

The subject matter of WO 2018 087261 A1 (PFEIFER & LANGEN) is a process for the synthesis of a simple sugar, preferably of D-allulose from an educt, preferably D-fructose under heterogeneous or homogeneous catalysis which incorporates chemical and/or enzymatic catalysis, wherein the synthesis is carried out in at least two reactors connected in series and the reaction product emerging from the first reactor is subjected to chromatographic separation before entering the second reactor. The chromatographic separation is preferably integrated into a simulated moving bed.

Typically, allulose is commercialized in the form of a syrup, obtainable by any of the above processes.

A further subject matter of the present invention relates to a process for producing crystalline allulose as described above, comprising or consisting of the following steps:

(a) Providing an aqueous allulose preparation, preferably an evaporator concentrate (“allulose syrup”), comprising or consisting of the following components, based on the dry matter contained in the preparation:

(i) about 90 to about 99% by weight allulose;

(ii) about 0.5 to about 10% by weight fructose;

(iii) 0 to about 5% by weight carbohydrates other than allulose and fructose; with the proviso that the quantities add up to 100% by weight;

(b) Optionally treating the allulose preparation of step (a) with a decolorizing agent;

(c) Concentrating the preparation from step (a) or step (b) until the saturation concentration is exceeded;

(d) Adding allulose seed crystals to the supersaturated preparation of step (c); and

(e) Cooling the preparation of step (c) at a rate of from 0.1 to about 1 K/h until a crystal content from about 30 to about 50% by weight is reached; and

(f) Separating and optionally drying the crystals.

As a matter of form, it should be pointed out that embodiments which result in more or less than 100% by weight are neither included in the invention nor covered by the claims. In any case, one skilled in the art is able, on the basis of the description and examples, to find compositions that fulfill the technical teaching without having to perform an inventive step.

In a preferred embodiment of the process according to the invention, the preparation of step (a) has an allulose content of ≥90% by weight, preferably ≥91% by weight, preferably ≥92% by weight, preferably ≥93% by weight, preferably ≥94% by weight, preferably ≥95% by weight, preferably ≥96% by weight, preferably ≥97% by weight, preferably ≥98% by weight, based on the dry matter contained in the preparation in each case.

In a further embodiment of the process according to the invention, the preparation of step (a) has an allulose content of ≤99% by weight, preferably ≤98% by weight, preferably ≤97% by weight, preferably ≤96% by weight, preferably ≤95% by weight, preferably ≤94% by weight, preferably ≤93% by weight, preferably ≤92% by weight, preferably ≤91% by weight, based on the dry matter contained in the preparation in each case.

As already mentioned, the preparation of step (a) has a fructose content from about 0.5 to about 10% by weight fructose, based on the dry matter contained in the preparation.

In a preferred embodiment of the process according to the invention, the preparation of step (a) has a fructose content from about 1 to about 10% by weight fructose, preferably from about 1 to about 3% by weight, and most preferably from about 1.5 to about 2.5% by weight, based on the dry matter contained in the preparation.

In a further embodiment of the process according to the invention, the preparation of step (a) has a fructose content from about 0.5 to about 1% by weight fructose, or from about 1 to about 1.5% by weight, or from about 1.5 to about 2.0% by weight, or from about 2.0 to about 2.5% by weight, or from about 2.5 to about 3.0% by weight, or from about 3.0 to about 3.5% by weight, or from about 3.5 to about 4.0% by weight, or from about 4.0 to about 4.5% by weight, or from about 4.5 to about 5.0% by weight, or from about 5.0 to about 5.5% by weight, or from about 5.5 to about 6.0% by weight, or from about 6.0 to about 6.5% by weight, or from about 6.5 to about 7.0% by weight, or from about 7.0 to about 7.5% by weight, or from about 7.5 to about 8.0% by weight, or from about 8.0 to about 8.5% by weight, or from about 8.5 to about 9.0% by weight, or from about 9.5 to about 10% by weight, based on the dry matter contained in the preparation in each case.

According to the invention, the preparations of step (a) have a solids content of from 55 to 98% by weight and preferably from about 75 to about 95% by weight.

In a further embodiment of the process according to the invention, the preparation of step (a) has a solids content of about 75% by weight, or of about 76% by weight, or of about 77% by weight, or of about 78% by weight, or of about 79% by weight, or of about 75% by weight, or of about 76% by weight, or of about 77% by weight, or of about 78% by weight, or of about 79% by weight, or of about 80% by weight, or of about 81% by weight, or of about 82% by weight, or of about 83% by weight, or of about 84% by weight, or of about 85% by weight, or of about 86% by weight, or of about 87% by weight, or of about 88% by weight, or of about 89% by weight, or of about 90% by weight, or of about 91% by weight, or of about 92% by weight, or of about 93% by weight, or of about 94% by weight, or of about 95% by weight.

In a further embodiment of the process according to the invention, the preparation of step (a) has a solids content from about 75 to about 80% by weight, from about 80 to about 85% by weight, from about 85 to about 90% by weight, or from about 90 to about 95% by weight.

Optionally, the preparations of step (a) can be treated with a decolorizing agent, for example activated charcoal or decolorizing resins (step (b) of the process). The decolorizing resins which are suitable for the purposes of the present application are commercially available and well known to those skilled in the art and therefore do not need to be explained in more detail.

In step (c), the preparations of step (a) or the decolored preparations of step (a) are concentrated until the saturation concentration is exceeded, which is implemented by a thermal evaporation step.

According to the present invention, various evaporators are suitable for carrying out the concentration step (c), for example steam boilers, thin film evaporators, falling film evaporators, boiler evaporators, coaxial evaporators, natural circulation evaporators, plate evaporators, or forced circulation evaporators. These devices are well known to those skilled in the art and therefore do not need to be explained in more detail. The concentration step typically takes place in either one or in two to three evaporators connected in series; preferably, falling film evaporators or plate evaporators are used in this case.

The concentration is usually carried out at a temperature from about 50 to about 75° C. If the process step is not carried out in one stage but in two or three stages, it has proven to be advantageous for energy reasons to exclude the temperature range between 59 and 71° C., i.e. to work in temperature ranges above and below this in the evaporators.

In step (d), seed crystals are added to the intermediate product obtained in step (c), wherein about 1 to about 3 and typically 1.5 to 2% by weight allulose crystals are used—based on the concentrates.

The supersaturation is then maintained by evaporation, but preferably by slow cooling (0.1-1 K/h), until a crystal content from about 30 to about 50% by weight is reached. The crystals are then separated off and optionally also dried.

A further subject matter of the present application is also of course allulose in crystalline form (as defined in paragraphs [0017]-[0020]), obtainable by means of the process described in paragraphs [0025]-[0040].

A further subject matter of the invention relates to the use of crystalline allulose as described in paragraphs [0017]-[0020] or obtainable through the process described in paragraphs [0025]-[0040] for the production of oral preparations.

Surprisingly, it was found that replacing table sugar with allulose crystals in the production of oral preparations has a positive influence on the sensory as well as physical and taste properties of the oral preparations, which are described in more detail below.

In particular, it was found that when table sugar is replaced by allulose, the a_{w} value of the ready-made products is reduced.

In a preferred embodiment, the aforementioned oral preparation is an oral preparation selected from the group formed by foods, oral and dental care agents, and pharmaceutical preparations.

### Food

As already mentioned, the crystalline allulose can be used as described in paragraphs [0017]-[0020] or is obtainable by means of the process described in paragraphs [0025]-[0040] for the production of food. For example and not exclusively, the following can be considered:

Beverages;

Baked goods;

Grain products;

Snack items;

Confectionery;

Dairy products;

Fruit preparations;

Condiments;

Vegetable preparations;

Meat products; or

Spices and spice mixes.

The group of beverages comprises both alcoholic and non-alcoholic products, such as coffee, tea, iced tea, wine, wine-based beverages, beer, beer-based beverages, liqueurs, schnapps, brandies, (carbonated) fruit-based lemonades, (carbonated) isotonic drinks and flavored mineral water, (carbonated) soft drinks, nectars, spritzers, fruit and vegetable juices, fruit or vegetable juice preparations, instant drinks, such as instant cocoa drinks, instant tea drinks, instant coffee drinks, or instant fruit drinks. Beverages with crystalline allulose according to the invention have, for example, the following advantages:

Improved fruitiness;

No negative influence on foam stability;

Improved mouthfeel compared to other reduced-calorie beverages;

Sweetener without calories and without an E number;

Real sugar without calories+without an “off-flavor”;

100% Sucrose replacement technically possible;

Suitable for diabetics;

Suitable for all forms (from instant beverages to beer mixes);

Transparent color in the end product;

Excellent synergies in combination with sucrose (sensory, haptic, optics);

Sucrose-like sweetness profile;

Increased color intensity, e.g. in orange lemonade;

Improved color stability;

Improved CO₂ retention after bottle is opened;

Improved tanginess and freshness;

Improved mouthfeel; as well as

Very good suitability for vending machine drinks (instant) due to faster solubility.

Baked goods, grain products, and snack items are also suitable. These include, for example, bread, dry cookies, cakes, muffins, donuts, and other pastries. Baked or deep-fried potato chips or potato dough products or corn-based or peanut-based extrudates, for example, are suitable as snack items. Breakfast cereals, granola bars, and pre-cooked ready-made rice products are examples of grain products.

Examples of confectionery products are chocolates, chocolate bar products, other bar products, fruit gummies, gum candies, hard and soft caramels, chewing gum, tablets, mints, coatings (fondants), jams, sherbets, fruit sauces, and fruit fillings. The products from these groups have at least one of the following advantages:

Sweetener without calories and without an E number;

Real sugar without calories+without an “off-flavor”;

Suitable as a humectant, prevents drying out, and extends the expiration date;

Makes product smoother/softer, does not dry out product like other sugar substitutes;

Improves the flavor and fruitiness of sugar acid coatings (e.g. fruit gummies);

Improved solubility in compressed and effervescent tablets;

Suitable in bars (cereal, protein bars);

Improved sensory profile through caramel notes;

Softer and moister crust compared to other sugars;

Improved dough viscosity;

100% Sucrose replacement possible;

Suitable for diabetics;

Reduced baking time due to increased reactivity of the allulose crystals;

Optimal browning result at reduced baking temperatures, fewer or no addition of dyes required;

Better processing capabilities of baking mixes;

Enhancement of umami flavor in snacks;

Enhancement of the caramel notes in toffees; as well as

Enhancement of the chocolate flavor and/or the cocoa notes in chocolates and products containing chocolate or cocoa.

Dairy products include, for example, milk drinks, buttermilk drinks, milk-based ice cream, yogurt, kefir, cream cheese, soft cheese, hard cheese, dried milk powder, whey, whey drinks, butter, buttermilk, products containing partially or fully hydrolyzed milk protein, products made from soy protein or other soybean fractions (e.g. soy milk and products made therefrom, fruit drinks with soy protein, preparations containing soy lecithin, fermented products such as tofu or tempeh or products made therefrom), and products from other plant-based protein sources, for example oat protein drinks. The products from these groups have at least one of the following advantages:

Sweetener without calories and without an E number;

Increased perception of sweetness in flavored milk drinks by combining crystalline allulose and sucrose (Maillard reaction) in amounts of 2-15% by weight;

Positive flavor enhancement in the presence of proteins;

Real sugar without calories+without an “off-flavor”;

100% Sucrose replacement technically possible;

Suitable for diabetics;

Improved flavor profile (e.g. caramel ice cream: no or reduced added flavor);

Lowering the freezing point and improving the melting behavior of ice cream;

Softer, creamier texture in yogurt and quark preparations, desserts, and cream cheese;

Increased sensory sensitivity to cold;

Intensified fruitiness in sorbets; as well as

Reduction of the negative cooking taste in common heating processes.

Examples of fruit preparations are jams, sherbets, fruit sauces, and fruit fillings. Honey and honey-like products, such as maple syrup, are also included within the meaning of the invention. The products from these groups have at least one of the following advantages:

Sweetener without calories and without an E number;

Real sugar without calories+without an “off-flavor”;

100% Sucrose replacement and any combination possible;

Suitable for diabetics;

Improved flavor profile;

More intense coloring;

Extended durability due to lower aw value;

Enhancement of flavor intensity (e.g. more intense fruitiness);

Better spreadability with fruit spreads;

Better creaminess in honey and honey-like products;

Better incorporation of fruit preparations in yogurt and quark preparations, desserts;

Consistent fruitiness with less added acid;

Less foaming; as well as

Production of preserving sugar possible without drying.

Suitable condiments and vegetable preparations are, for example, ketchup (especially spice, curry, and BBQ ketchup), sauces, dried vegetables, frozen vegetables, pre-cooked vegetables, and cooked vegetables. This also includes products based on fat and oil or emulsions of same (e.g. mayonnaise, tartar sauce, dressings), other ready-made meals and soups (e.g. dry soups, instant soups, pre-cooked soups), spices, spice mixes, and in particular seasonings which are used, for example, in the snack sector. The products from these groups have at least one of the following advantages:

Sweetener without calories and without an E number;

Real sugar without calories and without an “off-flavor”;

100% Sucrose replacement possible;

Suitable for diabetics;

Improved coloring;

Extended shelf life;

Enhancement of flavor intensity, e.g. more intense fruitiness of the tomato in ketchup);

Improved viscosity of ketchup; as well as

Enhancement of the umami flavor;

Good stabilizing properties in food emulsions;

Suitable meat products include, for example, ham, fresh sausage or raw sausage preparations, seasoned or marinated fresh or cured meat products.

The group also comprises spices and spice mixes and sweetener enhancers, flavor enhancers, and masking agents, optionally in encapsulated form.

Sweeteners in which the sweetening power thereof is enhanced and/or in which the unpleasant taste notes thereof (astringent, metallic, tarry, greasy) are covered or masked comprise, for example: sucrose/saccharose, trehalose, lactose, maltose, melezitose, raffinose, isomaltulose, lactulose, D-fructose, D-glucose, D-galactose, L-rhamnose, D-sorbose, D-mannose, D-tagatose, D-arabinose, L-arabinose, D-ribose, D-glyceraldehyde, or maltodextrin. Also suitable are herbal preparations containing these substances, for example based on sugar beets (Beta vulgaris subsp., sugar fractions, sugar syrup, molasses), sugar cane (Saccharum officinarum subsp., molasses, sugar cane syrup), maple syrup (Acer subsp.), or agaves (agave syrup).

Synthetic, i.e. usually enzymatically produced, starches or sugar hydrolysates (invert sugar, fructose syrup) also come into consideration;

Fruit concentrates (e.g. based on apples or pears);

Sugar alcohols (e.g. erythritol, threitol, arabitol, ribotol, xylitol, sorbitol, mannitol, dulcitol, lactitol);

Proteins (e.g. miraculin, monellin, thaumatin, curculin, brazzein);

Sweeteners (e.g. magap, sodium cyclamate, acesulfame K, neohesperidin dihydrochalcone, saccharin sodium salt, aspartame, superaspartame, neotame, alitame, sucralose, stevioside, rebaudioside, lugduname, carrelame, sucrononate, sucrooctate, monatin, phenylodulcin);

Sweet-tasting amino acids (e.g. glycine, D-leucine, D-threonine, D-asparagine, D-phenylalanine, D-tryptophne, L-proline);

Other sweet-tasting substances with low molecular weight, such as hernandulcin, dihydrochalcone glycosides, glycyrrhizin, glycyrrhetinic acid, the derivatives and salts thereof, extracts of licorice (Glycyrrhizza glabra subsp.), Lippia dulcis extracts, Momordica subsp. extracts; or

Individual substances such as Momordica grosvenori (Luo Han Guo) and the resulting mogrosides, Hydrangea dulcis or Stevia subsp. (e.g. Stevia rebaudiana) extracts, as explained in more detail below.

Rebaudiosides belong to the steviosides, the main components of the plant Stevia rebaudiana, which is also known as sweetweed or honeyweed.

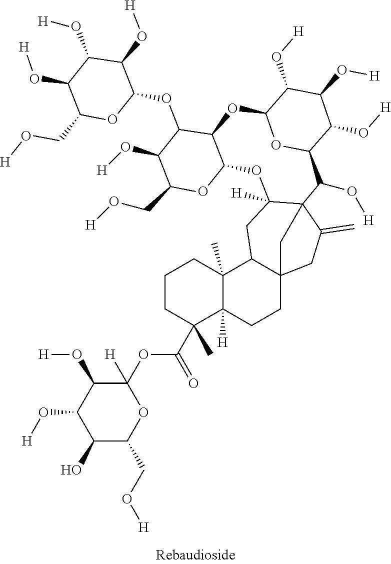 [SMILES: C=C1CC23CC[C@H]4[C@](C)(C(=O)OC5O[C@H](CO)[C@@H](O)[C@H](O)[C@H]5O)CCC[C@@]4(C)C2C[C@H]2O[C@@H]4O[C@@H](CO)[C@@H](O)C(O[C@@H]5O[C@H](CO)[C@@H](O)[C@H](O)[C@H]5O)[C@H]4O[C@@H]4O[C@H](C(O)C12C3)[C@@H](O)[C@H](O)[C@H]4O]

10% of the dry matter of the leaves is made up of the diterpene glycoid stevioside, followed by rebaudioside A (2 to 4% by weight), and ten other steviol glycosides, such as dulcoside. Rebaudioside and stevia extract are now approved sweeteners in most states; a daily intake of up to 4 mg stevioside per kilogram of body weight is considered harmless. According to the invention, individual rebaudiosides or the extracts of the stevia plant can be used. However, the use of rebaudioside A is particularly preferred, since this substance has less bitterness and the highest sweetening power.

As an alternative or in addition to the rebaudiosides or stevia extracts, various other active ingredients can also be used as sweeteners; they are:

Naringin;

Dihydrochalcones;

Mogrosides; as well as

Extracts of plants of the genus Rubus.

Naringin is a polyphenolic glycoside found in grapefruit and pomelo that gives them a bitter taste. The substance is known in particular for its lipid-lowering effect.

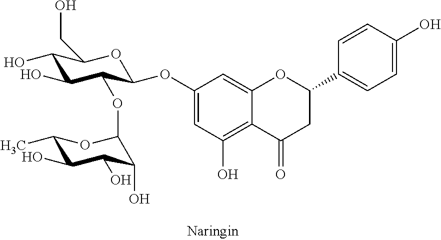

The dihydrochalcones are also polyphenols, wherein the two representatives dihydrochalcone and neohesperidin dihydrochalcone, which are known as artificial sweeteners, are particularly noteworthy:

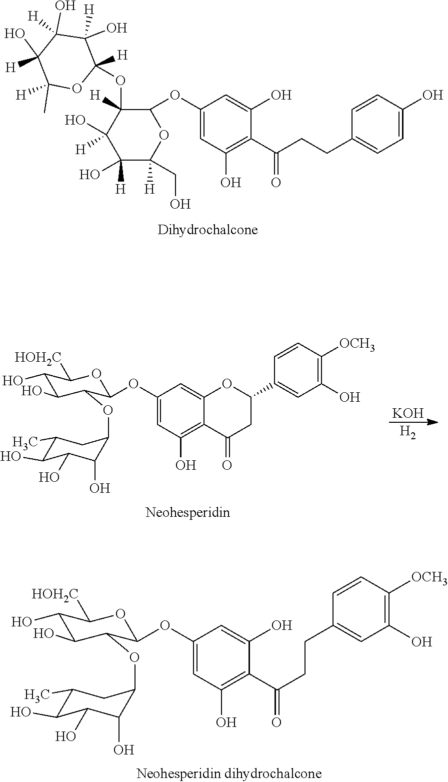

The term mogroside refers to a group of cucurbitane glycosides, which are known to be part of the natural sweetener Luo Han Guo. Of particular note here is mogroside V, which is 400 times sweeter than sugar.

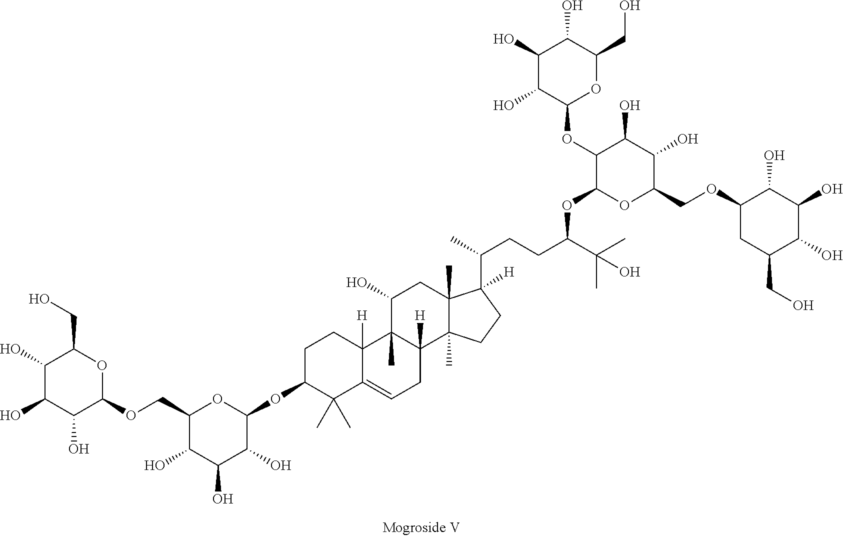

Finally, extracts of plants selected from the group formed by Rubus allegheniensis, Rubus arcticus, Rubus strigosus, Rubus armeniacus, Rubus caesius, Rubus chamaemorus, Rubus corylifolius agg., Rubus fruticosus agg., Rubus geoides, Rubus glaucus, Rubus gunnianus, Rubus idaeus, Rubus illecebrosus, Rubus laciniatus, Rubus leucodermis, Rubus loganobaccus, Rubus loxensis, Rubus nepalensis, Rubus nessensis, Rubus nivalis, Rubus odoratus, Rubus pentalobus, Rubus phoenicolasius, Rubus saxatilis, Rubus setchuenensis, Rubus spectabilis and Rubus ulmifolius, and mixtures thereof. These are essentially extracts from different types of blackberries and raspberries that contain rubosides. Extracts of Rubus suavissimus are preferred.

Another active ingredient in this group is glycyrrhizic acid or a corresponding salt or an extract containing this substance.

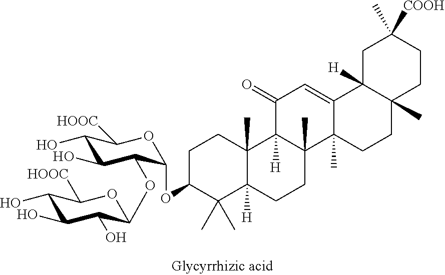

According to the invention, it is possible to use the acid itself, the salts thereof—for example the sodium, potassium, or ammonium salt—or the extracts of the plant Glycyrrhiza glabra. The monoammonium glycyrrhizate is particularly preferred.

Accordingly, a further subject matter of the present invention is the use of allulose, in particular crystalline allulose, as described in paragraphs [0017]-[0020] or obtainable through the process described in paragraphs [0025]-[0040] for enhancing the sweetening power and/or for covering up and/or for masking the unpleasant flavor notes (astringent, metallic, tarry, greasy) of sweeteners, particularly the sweeteners mentioned above.

In a particularly preferred embodiment, allulose, in particular crystalline allulose, is used as described in paragraphs [0017]-[0020] or is obtainable through the process described in paragraphs [0025]-[0040] for masking the unpleasant taste notes (astringent, metallic, tarry, greasy) of sweeteners, particularly the sweeteners mentioned above. In such use, the allulose/sweetener weight ratio ranges from about 10:90 to 90:10, preferably from about 25:75 to 75:25, and particularly from about 40:60 to 60:40.

Flavor enhancers, the effect of which is additionally enhanced and/or the unpleasant taste notes of which (astringent, metallic, tarry, greasy) are covered up or masked, comprise, for example: nucleotides (e.g. adenosine 5′-monophosphate, cytidine 5′-monophosphate), or the pharmaceutically acceptable salts thereof, lactisols, sodium salts (e.g. sodium chloride, sodium lactate, sodium citrate, sodium acetate, sodium gluconoate), other hydroxyflavanones (e.g. eriodictyol, homoeriodictyol, or the sodium salts thereof), in particular according to US 2002/0188019, hydroxybenzoic acid amides according to DE 10 2004 041 496 (e.g. 2,4-dihydroxybenzoic acid vanillylamide, 2,4-dihydroxybenzoic acid N-(4-hydroxy-3-methoxybenzyl)amide, 2,4,6-trihydroxybenzoic acid N-(4-hydroxy-3-methoxybenzyl)amide, 2-hydroxybenzoic aci d-N-(4-hydroxy-3-methoxybenzyl)amide, 4-hydroxybenzoic acid-N-(4-hydroxy-3-methoxybenzyl)amide, 2,4-dihydroxybenzoic acid-N-(4-hydroxy-3-methoxybenzyl)amide monosodium salt, 2,4-dihydroxybenzoic acid-N-2-(4-hydroxy-3-methoxyphenyl)ethylamide, 2,4-dihydroxybenzoic acid-N-(4-hydroxy-3-ethoxybenzyl)amide, 2,4-dihydroxybenzoic acid N-(3,4-dihydroxybenzyl)amide, and 2-hydroxy-5-methoxy-N-[2-(4-hydroxy-3-methoxyphenyl)ethyl]amide (aduncamide), 4-hydroxybenzoic acid vanillylamide), bitter-masking hydroxydeoxybenzoins, for example according to WO 2006/106023 (e.g. 2-(4-hydroxy-3-methoxyphenyl)-1-(2,4,6-trihydroxyphenyl)ethanone, 1-(2,4-dihydroxyphenyl)-2-(4-hydroxy-3-methoxyphenyl)ethanone, 1-(2-hydroxy-4-methoxyphenyl)-2-(4-hydroxy-3-methoxyphenyl)ethanone), amino acids (e.g. gamma-aminobutyric acid according to WO 2005/096841 for reducing or masking an unpleasant taste impression such as bitterness), malic acid glycosides according to WO 2006/003107, salty-tasting mixtures according to PCT/EP 2006/067120, diacetyl trimers according to WO 2006/058893, mixtures of whey proteins with lecithins, and/or bitter-masking substances such as gingerdione according to WO 2007/003527.

Accordingly, a further subject matter of the present invention is the use of allulose, in particular crystalline allulose, as described in paragraphs [0017]-[0020] or obtainable through the process described in paragraphs [0025]-[0040] for covering up and/or masking the unpleasant flavor notes (astringent, metallic, tarry, greasy) of flavor enhancers, particularly the flavor enhancers mentioned above.

In a particularly preferred embodiment, allulose, in particular crystalline allulose, is used as described in paragraphs [0017]-[0020] or is obtainable through the process described in paragraphs [0025]-[0040] for masking the unpleasant taste notes (astringent, metallic, tarry, greasy) of flavor enhancers, in particular the flavor enhancers mentioned above. In such use, the allulose/flavor enhancer weight ratio ranges from about 10:90 to 90:10, preferably from about 25:75 to 75:25, and particularly from about 40:60 to 60:40.

Preferred flavorings are those that cause a sweet olfactory impression, wherein the further flavoring(s) that cause a sweet olfactory impression are preferably selected from the group consisting of: vanillin, ethyl vanillin, ethyl vanillin isobutyrate (=3-ethoxy-4-isobutyryloxybenzaldehyde), furaneol® (2,5-dimethyl-4-hydroxy-3(2H)-furanone), and derivatives (e.g. homofuraneol, 2-ethyl-4-hydroxy-5-methyl-3(2H)-furanone), homofuronol (2-ethyl-5-methyl-4-hydroxy-3(2H)-furanone, and 5-ethyl-2-methyl-4-hydroxy-3(2H)-furanone), maltol, and derivatives (e.g. ethyl maltol), coumarin, and derivatives, gamma-lactones (e.g. gamma-undecalactone, gamma-nonalactone), delta-lactones (e.g. 4-methyldeltalactone, massoilactone, delta-decalactone, tuberolactone), methylsorbate, divanillin, 4-hydroxy-2(or 5)-ethyl-5(or 2)-methyl-3(2H)-furanone, 2-hydroxy-3-methyl-2-cyclopentenone, 3-hydroxy-4,5-dimethyl-2(5H)-furanone, fruit esters and fruit lactones (e.g. n-butyl acetate, isoamyl acetate, ethyl propionate, ethyl butyrate, n-butyl butyrate, isoamyl butyrate, ethyl 3-methylbutyrate, ethyl n-hexanoate, allyl n-hexanoate, n-butyl n-hexanoate, ethyl n-octanoate, ethyl 3-methyl-3-phenylglycidate, ethyl 2-trans-4-cis-decadienoate), 4-(p-hydroxyphenyl)-2-butanone, 1,1-dimethoxy-2,2,5-trimethyl-4-hexane, 2,6-dimethyl-5-hepten-1-al, 4-hydroxycinnamic acid, 4-methoxy-3-hydroxycinnamic acid, 3-methoxy-4-hydroxycinnamic acid, 2-hydroxycinnamic acid, 2,4-dihydroxybenzoic acid, 3-hydroxybenzoic acid, 3,4-dihydroxybenzoic acid, vanillic acid, homovanillic acid, vanillomandelic acid, and phenylacetaldehyde.

Accordingly, a further subject matter of the present invention is the use of allulose, in particular crystalline allulose, as described in paragraphs [0017]-[0020] or obtainable through the process described in paragraphs [0025]-[0040] for enhancing the sweetening power and/or for covering up and/or for masking the unpleasant taste notes (astringent, metallic, tarry, greasy) of flavorings, particularly the flavorings mentioned above.

In a particularly preferred embodiment, allulose, in particular crystalline allulose, is used as described in paragraphs [0017]-[0020] or is obtainable through the process described in paragraphs [0025]-[0040] for masking the unpleasant taste notes (astringent, metallic, tarry, greasy) of flavorings, particularly the flavorings mentioned above. In such use, the allulose/flavor enhancer weight ratio ranges from about 10:90 to 90:10, preferably from about 25:75 to 75:25, and particularly from about 40:60 to 60:40.

### Oral and Dental Care Agents

As already mentioned, the crystalline allulose can be used as described in paragraphs [0017]-[0020] or is obtainable by means of the process described in paragraphs [0025]-[0040] for the production of oral and dental care agents. Examples of this are toothpastes, tooth gels, tooth powder, mouthwashes, (medicinal) chewing gum, and the like. The products from these groups have at least one of the following advantages:

Sweetener without calories and without an E number;

Real sugar without calories+without an “off-flavor”;

100% Sucrose replacement and any combination possible;

Suitable for diabetics;

Improved flavor profile;

Improved coloring;

Enhancement of flavor intensity (e.g. more intense mint flavor);

Enhancement of the cooling effect (e.g. longer-lasting menthol taste); as well as

Less foaming.

Toothpastes or dental creams are generally understood to be gel-like or pasty preparations made from water, thickeners, humectants, grinding or cleaning agents, surfactants, sweeteners, flavorings, active deodorizing ingredients, and active ingredients to protect against oral and dental diseases. In the toothpastes according to the present invention, all the usual cleaning agents can be used, such as chalk, dicalcium phosphate, insoluble sodium metaphosphate, aluminum silicates, calcium pyrophosphate, finely divided synthetic resins, silica, aluminum oxide, and aluminum oxide trihydrate.

Preferred cleaning agents for the toothpastes according to the present invention are primarily finely divided silica xerogel, silica hydrogel, precipitated silica, alumina trihydrate, and finely divided alpha aluminum oxide, or mixtures of these cleaning agents in quantities of 15 to 40% by weight of the toothpaste. Low-molecular-weight polyethylene glycols, glycerol, sorbitol, or mixtures of these products in amounts of up to 50% by weight are primarily suitable as humectants. Among the known thickeners, the thickening, finely divided silica gels and hydrocolloids are suitable, such as carboxymethyl cellulose, hydroxyethyl cellulose, hydroxypropyl guar, hydroxyethyl starch, polyvinylpyrrolidone, high-molecular-weight polyethylene glycol, plant gums such as tragacanth, agar-agar, carrageen moss, gum arabic, xanthan gum, and carboxyvinyl polymers (e.g. Carbopol® types). In addition to the mixtures of menthofuran and menthol compounds, the oral and dental care agents can in particular contain surface-active substances, preferably anionic and nonionic high-foaming surfactants, such as the substances already mentioned above, but particularly alkyl ether sulfate salts, alkyl polyglucosides, and mixtures thereof.

Other common toothpaste additives include:

Preservatives and antimicrobials such as methyl, ethyl or propyl p-hydroxybenzoate, sodium sorbate, sodium benzoate, bromochlorophene, phenylsalicylic acid ester, thymol, and the like;

Active antitartar ingredients, e.g. organophosphates such as 1-hydroxyethane-1,1-diphosphonic acid, 1-phosphonopropane-1,2,3-tricarboxylic acid and others, which are known, for example, from U.S. Pat. No. 3,488,419, DE 2224430 A1 and DE 2343196 A1;

Other caries-inhibiting substances such as sodium fluoride, sodium monofluorophosphate, tin fluoride;

Sweeteners such as sucrose heparin sodium, sodium cyclamate, sucrose, lactose, maltose, fructose, or Aspartame® (L-aspartyl-L-phenylalanine methyl ester), stevia extracts, or the sweetening components thereof, in particular ribeaudiosides;

Additional flavors such as eucalyptus oil, anise oil, fennel oil, caraway oil, methyl acetate, cinnamaldehyde, anethole, vanillin, thymol, and mixtures of these and other natural and synthetic flavors;

Pigments such as titanium dioxide;

Dyes;

Buffer substances such as primary, secondary, or tertiary alkali metal phosphates, or citric acid/sodium citrate;

Wound-healing and anti-inflammatory substances such as allantoin, urea, azulene, active chamomile ingredients, and acetylsalicylic acid derivatives.

A preferred embodiment of the oral preparations are toothpastes in the form of an aqueous, pasty dispersion containing polishing agents, humectants, viscosity regulators, and optionally other customary components, as well as the mixture of menthofuran and menthol compounds in amounts of 0.5 to 2% by weight.

In mouthwashes, a combination with aqueous-alcoholic solutions of various degrees of essential oils, emulsifiers, astringent and toning drug extracts, anti-tartar, antibacterial additives, and taste correctors is easily possible. Another preferred embodiment of the invention is a mouthwash in the form of an aqueous or aqueous-alcoholic solution containing the mixture of menthofuran and menthol compounds in amounts of 0.5 to 2% by weight. In mouthwashes that are diluted before use, sufficient effects can be achieved with higher concentrations, depending on the intended dilution ratio.

Hydrotropes, such as ethanol, isopropyl alcohol, or polyols, can also be used to improve the flow behavior; these substances largely correspond to the carriers described at the outset. Polyols which are included herein preferably have 2 to 15 carbon atoms and at least two hydroxyl groups. The polyols can also contain other functional groups, in particular amino groups, or be modified with nitrogen. Typical examples include

Glycerol;

Alkylene glycols such as ethylene glycol, diethylene glycol, propylene glycol, butylene glycol, hexylene glycol, and polyethylene glycols having an average molecular weight of 100 to 1,000 daltons;

Technical oligoglycerol mixtures with an inherent degree of condensation of 1.5 to 10 such as technical diglycerol mixtures with a diglycerol content of from 40 to 50% by weight;

Methylol compounds such as, in particular, trimethylolethane, trimethylolpropane, trimethylolbutane, pentaerythritol, and dipentaerythritol;

Lower alkyl glucosides, particularly those having 1 to 8 carbons in the alkyl radical, such as methyl and butyl glucoside;

Sugar alcohols having 5 to 12 carbon atoms, such as sorbitol or mannitol;

Sugars having 5 to 12 carbon atoms, such as glucose or sucrose;

Amino sugars such as glucamine;

Dialcohol amines such as diethanolamine or 2-amino-1,3-propanediol.

Examples of suitable preservatives are phenoxyethanol, formaldehyde solution, parabens, pentanediol, or sorbic acid, as well as the silver complexes known under the name Surfacine® and the other classes of substances listed in Annex 6, Parts A and B of the Cosmetics Ordinance.

Perfume oils to be considered are mixtures of natural and synthetic fragrances. Natural fragrances are extracts of flowers (lily, lavender, rose, jasmine, neroli, ylang-ylang), stems and leaves (geranium, patchouli, petitgrain), fruits (aniseed, coriander, caraway, juniper), fruit rind (bergamot, lemon, oranges), roots (mace, angelica, celery, cardamom, costus, iris, calamus), wood (pine, sandalwood, guaiac, cedar, rosewood), herbs and grasses (tarragon, lemongrass, sage, thyme), needles and twigs (spruce, fir, pine, mountain pine), and resins and balms (galbanum, elemi, benzoin, myrrh, olibanum, opoponax). Furthermore, animal-based raw materials are included, such as civet and castoreum. Typical synthetic fragrance compounds are products of the ester, ether, aldehyde, ketone, alcohol, and hydrocarbon type. Fragrance compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethyl methylphenylglycinate, allyl cyclohexyl propionate, styrallyl propionate, and benzyl salicylate. The ethers include, for example, benzyl ethyl ether; the aldehydes include, for example, the linear alkanals having 8 to 18 carbon atoms, citral, citronellal, citronellyloxyacetaldehyde, cyclamen aldehyde, hydroxycitronellal, lilial, and bourgeonal; the ketones include, for example, the ionones, α-isomethylionone, and methylcedryl ketone; the alcohols include anethole, citronellol, eugenol, isoeugenol, geraniol, linalool, phenylethyl alcohol, and terpineol; and the hydrocarbons mainly include terpenes and balms. However, preference is given to using mixtures of different fragrances which together produce an appealing fragrance note. Essential oils of lower volatility, which are usually used as flavor components, are also suitable as perfume oils, for example sage oil, chamomile oil, clove oil, lemon balm oil, mint oil, cinnamon leaf oil, lime blossom oil, juniper berry oil, vetiver oil, olibanum oil, galbanum oil, labolanum oil, and lavandin oil. Preferable for use are bergamot oil, dihydromyrcenol, lilial, lyral, citronellol, phenylethyl alcohol, α-hexyl cinnamaldehyde, geraniol, benzylacetone, cyclamen aldehyde, linalool, Boisambrene Forte, Ambroxan, indole, Hedione, Sandelice, lemon oil, mandarin oil, orange oil, allylamyl glycolate, cyclovertal, lavandin oil, clary sage oil, β-damascone, bourbon geranium oil, cyclohexyl salicylate, Vertofix Coeur, Iso E Super, Fixolide NP, Evernyl, Iraldein Gamma, phenylacetic acid, geranyl acetate, benzyl acetate, rose oxide, Romilllat, Irotyl, and Floramat, alone or in mixtures.

Examples of flavors that can be used are peppermint oil, spearmint oil, anise oil, star anise oil, caraway oil, eucalyptus oil, fennel oil, lemon oil, wintergreen oil, clove oil, menthol, and the like.

The preferred oral preparations can also be chewing gums. These products typically contain a water-insoluble and a water-soluble component.

The water-insoluble base, which is also referred to as “gum base,” usually comprises natural or synthetic elastomers, resins, fats and oils, softeners, fillers, dyes, and possibly waxes. The portion of the base in the overall composition is usually from 5 to 95% by weight, preferably from 10 to 50% by weight, and in particular from 20 to 35% by weight. In a typical embodiment of the invention, the base consists of 20 to 60% by weight synthetic elastomers, 0 to 30% by weight natural elastomers, 5 to 55% by weight softeners, 4 to 35% by weight fillers, and minor amounts of additives such as dyes, antioxidants, and the like together, with the proviso that they are water-soluble at most in small amounts.

Examples of suitable synthetic elastomers are polyisobutylenes with average molecular weights (according to GPC) of 10,000 to 100,000 and preferably 50,000 to 80,000, isobutylene isoprene copolymers (“butyl elastomers”), styrene butadiene copolymers (styrene/butadiene ratio of e.g. 1:3 to 3:1), polyvinyl acetates with average molecular weights (according to GPC) of 2,000 to 90,000 and preferably 10,000 to 65,000, polyisoprenes, polyethylene, vinyl acetate/vinyl laurate copolymers, and mixtures thereof. Examples of suitable natural elastomers are rubbers such as smoked or liquid latex or guayule and natural gums such as jelutong, leche caspi, perillo, sorva, massaranduba balata, massaranduba chocolate, loquat, rosindinba, chicle, gutta hang kang, and mixtures thereof. The choice of synthetic and natural elastomers and the mixing ratios thereof essentially depends on whether bubble gums are to be created with the chewing gum or not. Elastomer mixtures containing jelutong, chicle, sorva, and massaranduba are preferably used.

In most cases, the elastomers turn out to be too hard or insufficiently deformable during processing such that it has proven to be advantageous to use special softeners, which of course also have to meet all the requirements for approval as food additives. Particularly suitable in this respect are esters of resin acids, for example esters of lower aliphatic alcohols or polyols with fully or partially hydrogenated, monomeric or oligomeric resin acids. In particular, the methyl, glycerol, or pentareythritol esters and mixtures thereof are used for this purpose. Alternatively, terpene resins can also be considered, which can be derived from alpha-pinene, beta-pinene, delta-limonene, or mixtures thereof.

The fillers or texturing agents can be magnesium or calcium carbonate; ground pumice stone; silicates, especially magnesium or aluminum silicates; clays; aluminum oxides; talc; titanium dioxide; monocalcium, dicalcium, and tricalcium phosphate; and cellulosic polymers.

Suitable emulsifiers are tallow, hydrogenated tallow, hydrogenated or partially hydrogenated vegetable oils, cocoa butter, partial glycerides, lecithin, triacetin, and saturated or unsaturated fatty acids having 6 to 22 and preferably 12 to 18 carbon atoms, and mixtures thereof

Dyes and whitening agents that can be used include the FD and C types approved for coloring food, plant and fruit extracts, and titanium dioxide.

The base compounds can contain waxes or be wax-free; examples of wax-free compositions can be found, inter alia, in U.S. Pat. No. 5,286,500, the content of which is hereby expressly incorporated by reference.

In addition to the water-insoluble gum base, chewing gum preparations regularly contain a water-soluble portion, for example softeners, sweeteners, fillers, flavorings, flavor enhancers, emulsifiers, dyes, acidifiers, antioxidants, and the like are formed, here with the proviso that the ingredients have at least sufficient water solubility. Depending on the water solubility of the specific representatives, individual components can therefore belong both to the water-insoluble and to the water-soluble phase. However, it is also possible to use combinations, for example of a water-soluble and a water-insoluble emulsifier, wherein the individual representatives are then in different phases. Usually, the water-insoluble portion constitutes from 5 to 95% and preferably from 20 to 80% by weight of the preparation.

Water-soluble softeners or plasticizers are added to chewing gum compositions to improve chewability and chewing sensation and are typically present in the mixtures at levels of from 0.5 to 15% by weight. Typical examples are glycerol, lecithin, and aqueous solutions of sorbitol, hydrogenated starch hydrolysates, or corn syrup.

Additional sweeteners, in addition to the crystalline allulose, are both sugar-containing and sugar-free compounds, which are used in amounts of from 5 to 95%, preferably 20 to 80%, and in particular 30 to 60% by weight, based on the chewing gum composition. Typical sucrose sweeteners are sucrose, dextrose, maltose, dextrin, dried invert sugar, fructose, levulose, galactose, corn syrup, and mixtures thereof. Possible sugar substitutes are sorbitol, mannitol, xylitol, hydrogenated starch hydrolysates, maltitol, and mixtures thereof. Also suitable as additives are so-called HIAS's (High-Intensity Artificial Sweeteners), such as sucralose, aspartame, acesulfame salts, alitame, sucrose and sucrose salts, cyclamic acid and the salts thereof, glycyrrhizin, dihydrochalcone, thaumatin, monellin, and the like, alone or in mixtures. The hydrophobic HIAS's, which are the subject matter of international patent application WO 2002 091849 A1 (Wrigleys), and stevia extracts and the active components thereof, in particular ribeaudioside A, are also particularly effective. The amount used of these substances depends primarily on their performance and is typically in the range of from 0.02 to 8% by weight.

Fillers such as polydextrose, raftilose, rafitilin, fructooligosaccharides (NutraFlora), palatinose oligosaccharides, guar gum hydrolysates (Sunfiber), and dextrins are particularly suitable for the production of low-calorie chewing gum.

The choice of other flavorings is practically unlimited and not critical to the essence of the invention. Typically, the total amount of all flavorings is from 0.1 to 15% and preferably from 0.2 to 5% by weight of the chewing gum composition. Other suitable flavorings are, for example, essential oils, synthetic flavors, and the like, such as anise oil, star anise oil, caraway oil, eucalyptus oil, fennel oil, lemon oil, wintergreen oil, clove oil, and the like, as they are also used in oral and dental care agents, for example.

The chewing gums can also contain auxiliaries and additives that are suitable, for example, for dental care, specifically for combating plaque and gingivitis, such as chlorhexidine, CPC, or trichlosan. Furthermore, pH regulators (e.g. buffers or urea), active ingredients effective against caries (e.g. phosphates or fluorides), and biogenic active ingredients (antibodies, enzymes, caffeine, plant extracts) can be included, as long as these substances are approved for food and do not interact with each other in an undesirable way.

### Pharmaceutical Agents

As already mentioned, the crystalline allulose can be used as described in paragraphs [0017]-[0020] or is obtainable by means of the process described in paragraphs [0025]-[0040] for the production of pharmaceutical agents. The suitable pharmaceutical agents comprise solid, tableted, or coated active ingredients and active ingredient mixtures, as well as liquid products. These products are characterized above all by the fact that they are not cariogenic and are therefore also suitable for diabetics. No crystallization occurs in liquid formulations. Tableting is easier, and tablets with allulose, such as AspirinPlus C, can also be taken without water due to their higher solubility.

According to the invention, in a first embodiment, (lozenge) tablets and coated tablets, i.e. solid forms of administration, are addressed which contain one or more active pharmaceutical ingredients together with a tableting agent and optionally a disintegrant, the nature of which, however, is not critical.

The invention relates to the surprising finding that the unpleasant, astringent, and/or metallic aftertaste common to many active pharmaceutical ingredients (particularly the so-called non-steroidal anti-inflammatory drugs, NSAIDs) and which makes them difficult to ingest is covered up and/or masked by the crystalline allulose. Without being conclusive, suitable active ingredients to which this finding applies are the following:

(1) ASPIRIN (acetylsalicylic acid)

(2) MINOXIDIL (6-piperidin-1-yl pyrimidine-2,4-diamine-3-oxide)

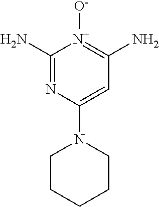

(3) ERYTHROMICIN

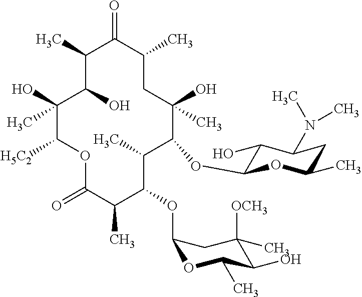

(4) DIMETINDENE (FENISTIL) (RS-dimethyl(2-(3-[pyridin-2-yl)ethyl]-1H-inden-2-yl)ethyl)amine)

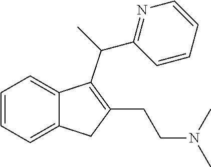 [SMILES: CC(C1=C(CCN(C)C)Cc2ccccc21)c1ccccn1]

(5) BETAMETHOSONE (8S,9R,10S,11S,13S,14S,16S,17R)-9-fluoro-11,17-(2-hydroxyacetyl)-10,13,16-trimethyl-6,7,8, 9,10,11,12,13,14,15,16,17-dodecahydro-3H-cyclopenta(alpha)-phenanthrene-3-one

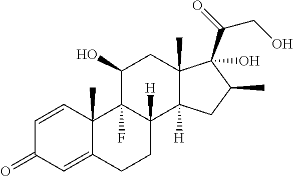

(6) IBUPROFEN (RS)-2-(4-(2-methylpropyl)phenyl)propanoic acid)

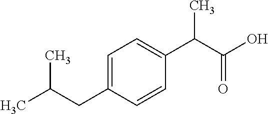

(7) KETOPROFEN (RS)2-(3-benzoylphenyl)propionic acid)

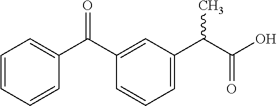 [SMILES: CC(C(=O)O)c1cccc(C(=O)c2ccccc2)c1]

(8) DICLOFENAC

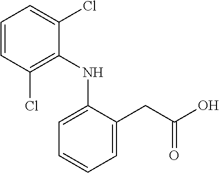 [SMILES: O=C(O)Cc1ccccc1Nc1c(Cl)cccc1Cl]

(9)Metronidazole (2-(2-methyl-5-nitro-1H-imidazol-1-yl)ethanol)

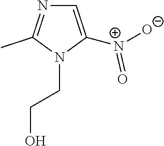

(10) ACYCLOVIR (2-amino-1,9-dihydro-94(2-hydroxyethoxy)methyl)-6H-purin-6-one),

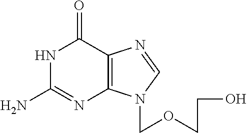 [SMILES: Nc1nc2c(ncn2COCCO)c(=O)[nH]1]

(11) IMIQUIMOD (3-(2-methylpropyl)-3,5,8-triazatricyclo[7.4.0.0.^{2,6}]trideca-1(9),2(6),4,7,10,12-hexaen-7-amine

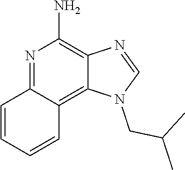 [SMILES: CC(C)Cn1cnc2c(N)nc3ccccc3c21]

(12) TERBINAFIN [(2E)-6,6-dimethylhept-2-en-4-yn-1-yl](methyl)(naphthalen-1-ylmethyl)amine)

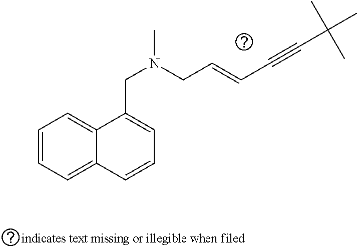 [SMILES: CN(C/C=C/C#CC(C)(C)C)Cc1cccc2ccccc12]

(13) CICLOPYRAXOLAMINE (6-cyclohexyl-1-hydroxy-4-methylpyridin-2(1H)-one)

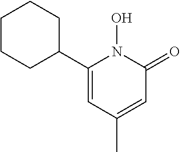

Accordingly, a further subject matter of the present invention is the use of allulose, particularly crystalline allulose, as described in paragraphs [0017]-[0020] or obtainable through the process described in paragraphs [0025]-[0040] for covering up and/or masking the unpleasant, astringent, and/or metallic aftertaste of active pharmaceutical ingredients.

In a particularly preferred embodiment, allulose, in particular crystalline allulose, is used as described in paragraphs [0017]-[0020] or is obtainable through the process described in paragraphs [0025]-[0040] for masking the unpleasant, astringent, and/or metallic aftertaste of the so-called non-steroidal anti-inflammatory drugs (NSAIDs).

In a further particularly preferred embodiment, allulose, in particular crystalline allulose, is used as described in paragraphs [0017]-[0020] or is obtainable through the process described in paragraphs [0025]-[0040] for masking the unpleasant, astringent, and/or metallic aftertaste of active ingredients selected from the group consisting of ASPIRIN (acetylsalicylic acid), MINOXIDIL (6-piperidin-1-ylpyrimidin-2,4-diamine-3-oxide), ERYTHROMICIN, DIMETINDENE (FENISTIL) (RS-dimethyl(2-(3-[pyridin-2-yl)ethyl]-1H-inden-2-yl)ethyl)amine), BETAMETHOSONE (8S,9R,10S,11S,13S,14S,16S,17R)-9-fluoro-11,17-(2-hydroxyacetyl)-10,13,16-trimethyl-6,7,8,9,10,11,12,13,14,15,16,17-dodecahydro-3H-cyclopenta(alpha)-phenanthrene-3-one, IBUPROFEN (RS)-2-(4-(2-methylpropyl)phenyl)propanoic acid), KETOPROFEN (RS)2-(3-benzoylphenyl)propionic acid), DICLOFENAC, Metronidazole (2-(2-methyl-5-nitro-1H-imidazol-1-yl)ethanol, ACYCLOVIR (2-amino-1,9-dihydro-9-((2-hydroxyethoxy)methyl)-6H-purin-6-one), IMIQUIMOD (3-(2-methylpropyl)-3,5,8-triazatricyclo[7.4.0.0.2,6]trideca-1(9),2(6),4,7,10,12-hexaen-7-amine, TERBINAFIN [(2E)-6,6-dimethylhept-2-en-4-yn-1-yl](methyl)(naphthalen-1-ylmethyl)amine), CICLOPYRAXOLAMINE (6-cyclohexyl-1-hydroxy-4-methylpyridin-2(1H)-one), or mixtures thereof.

In one of the previously mentioned uses, the allulose/active ingredient weight ratio ranges from about 10:90 to 90:10, preferably from about 25:75 to 75:25, and particularly about 40:60 to 60:40.

In addition to the solid preparations, liquid products can also be made using the crystalline allulose as described in paragraphs [0017]-[0020] or obtainable through the process described in paragraphs [0025]-[0040], such as cough syrup or spray formulations.

### EXAMPLES

The present invention will be more readily understood with reference to the following examples.

However, these examples are only intended to illustrate the invention and should not be construed as limiting the protected scope of the invention. Unless otherwise stated, all quantities are to be understood as % by weight.

### Example 1

### (According to the Invention): Crystallization of Allulose Solutions

1% by weight activated carbon was added to 1,000 liters of an aqueous allulose preparation with a dry matter content of 70% by weight and an allulose content of 92% by weight and a fructose content of 1.8% by weight, based on the dry matter contained in the preparation, and the mixture was stirred for 3 hours.

Then the decolorized solution was filtered and concentrated until a supersaturated solution was obtained.

This solution was transferred to a crystallizer and seeded with 1.5% by weight allulose crystals. The filtrate was then cooled at a rate of 1 K/h until the first crystals precipitated. The crystallization rate was reduced to 0.5 K/h until a crystal quantity of about 45% by weight was reached, based on the dry matter of the supersaturated solution.

The crystals were then separated and dried; the mother liquor was returned to the process. A crystalline allulose was obtained which had the following particle size distribution:

| | Dispersion pressure | | | |
|---|---|---|---|---|
| Parameter | [kPa] | X_{c(min)} | X_{Fe(max)} | Xₐᵣₑₐ |
| Minimum d10 [μm] | 5 | 72 | 105 | 90 |
| Minimum d50 [μm] | 5 | 150 | 230 | 190 |
| Minimum d90 [μm] | 5 | 235 | 350 | 270 |
| Minimum d10 [μm] | 20 | 55 | 75 | 75 |
| Minimum d50 [μm] | 20 | 125 | 220 | 195 |
| Minimum d90 [μm] | 20 | 225 | 335 | 280 |
| Minimum d10 [μm] | 460 | 12 | 10 | 12 |
| Minimum d50 [μm] | 460 | 45 | 70 | 55 |
| Minimum d90 [μm] | 460 | 135 | 175 | 135 |

### Comparative Example V1

### Crystallization of Allulose Solutions

1% by weight activated carbon was added to 1,000 liters of an aqueous allulose preparation with a dry matter content of 70% by weight and an allulose content of 92% by weight and a fructose content of 1.8% by weight, based on the dry matter contained in the preparation, and the mixture was stirred for 3 hours.

Then the decolorized solution was filtered and concentrated until a supersaturated solution was obtained.

This solution was transferred to a crystallizer. The filtrate was then cooled at a rate of 5 K/h until the first crystals precipitated. The crystallization rate was reduced to 1 K/h until a crystal quantity of about 45% by weight was reached, based on the dry matter of the supersaturated solution.

The crystals were then separated and dried; the mother liquor was returned to the process. A crystalline allulose was obtained which had the following particle size distribution:

| | Dispersion pressure | | | |
|---|---|---|---|---|
| Parameter | [kPa] | X_{c(min)} | X_{Fe(max)} | Xₐᵣₑₐ |
| Minimum d10 [μm] | 5 | 60 | 90 | 75 |
| Minimum d50 [μm] | 5 | 130 | 210 | 175 |
| Minimum d90 [μm] | 5 | 215 | 320 | 255 |
| Minimum d10 [μm] | 20 | 40 | 60 | 50 |
| Minimum d50 [μm] | 20 | 110 | 185 | 170 |
| Minimum d90 [μm] | 20 | 210 | 315 | 250 |
| Minimum d10 [μm] | 460 | 2 | 1 | 2 |
| Minimum d50 [μm] | 460 | 30 | 55 | 40 |
| Minimum d90 [μm] | 460 | 110 | 150 | 110 |

### Example 2, Comparative Examples V2 and V3

### Determination of the Water Value

Baked goods (brownies) of the same type were produced using sucrose (V2), crystalline allulose according to the invention as in Example 1, and crystalline according to Comparative Example V1. Samples of 20 g each of the three products were placed in containers, hermetically sealed, and then stored at 20° C. for 3 hours. A hygrometer was then used to determine the humidity in the gas space above the samples. The composition of the products and the humidity values are given in Table A below.

TABLE A
Composition and humidity

| Composition | 2 | | V2 | V3 | |
|---|---|---|---|---|---|
| Whole egg | 240.0 | g | 240.0 g | 240.0 | g |
| Salt | 2.0 | g | 2.0 g | 2.0 | g |
| Allulose according | 260.0 | g | — | — | |
| to Example 1 | | | | | |
| Allulose according | — | | 260.0 g | — | |
| to Example V1 | | | | | |
| Sucrose | — | | — | 260.0 | g |
| Butter | 230.0 | g | 230.0 g | 230.0 | g |
| Flour type 405 | 70.0 | g | 70.0 g | 70.0 | g |
| Cocoa powder | 110.0 | g | 110.0 g | 110.0 | g |
| Flavor (vanilla) | 5.0 | g | 5.0 g | 5.0 | g |

| | | | |
|---|---|---|---|
| Humidity [%] | | | |
| after 6 hours | 72 | 76 | 80 |
| after 48 hours | 71 | 75 | 75 |

The examples and comparative examples show that, with the new allulose quality, the water value in food can be reduced and thus the shelf life thereof can be improved.

### Example 3

### Taste Characteristics of Soft Drinks

Various substance mixtures based on steviol glycosides (rebaudioside A), with and without allulose crystals, were used to produce simple soft drinks, and the products were stored at 20° C. for 48 hours. Then the taste characteristics (descriptors: initial sweetness, sweetness intensity, sugary taste/mouthfeel) were rated by a panel of 8 trained testers on a line scale from 0 (none) to 10 (strongly pronounced). The compositions and results are summarized in Table 1 below. Embodiments 3 and 4 are according to the invention, while examples V4 to V6 are used for comparison. Example C corresponds to the standard, i.e. the taste assessment of the product without the addition of stevia.

TABLE 1
Taste properties of soft drink formulations

| Composition | C | 3 | 4 | V4 | V5 | V6 |
|---|---|---|---|---|---|---|
| Allulose | — | 7.0 | 10.0 | — | — | — |
| Sucrose | 10.0 | — | — | 7.0 | 8.0 | 10.0 |
| Citric acid | 0.2 | 0.2 | 0.2 | 0.2 | 0.2 | 0.2 |
| Rebaudioside A | — | 0.0045 | 0.0045 | 0.0045 | 0.0045 | 0.0045 |
| Lemon flavor | 0.1 | 0.1 | 0.1 | 0.1 | 0.1 | 0.1 |
| Water | Remainder up to 100 | | | | | |
| Taste assessment | | | | | | |
| Initial sweetness | 3.0 | 5.5 | 5.6 | 3.2 | 3.3 | 4.2 |
| Sweetness | 3.1 | 6.3 | 6.4 | 3.5 | 3.8 | 4.5 |
| intensity | | | | | | |
| Sugary taste and | 3.0 | 5.9 | 5.5 | 3.0 | 3.3 | 3.5 |
| mouthfeel | | | | | | |
| Bitterness in the | 0.3 | 0.2 | 0.2 | 0.5 | 0.4 | 0.2 |
| aftertaste | | | | | | |

The two preparations according to the invention show advantages in terms of initial sweetness, sweetness intensity, sugary taste, mouthfeel, and bitterness in the aftertaste, both compared to the standard and the stevia-containing preparations with sucrose.

The invention is illustrated below using further formulation examples. The allulose is a crystalline product that meets the preferred selection rules for particle size distribution—as described above—and is available from Savanna Ingredients GmbH.

### Formulation Example 1

### Soft Drinks

| Composition | 1.1 | 1.2 | 1.3 | 1.4 | 1.5 | 1.6 | 1.7 |
|---|---|---|---|---|---|---|---|
| Allulose | 10.0 | 10.0 | 7.0 | — | 10.0 | 8.0 | 7.0 |
| Rebaudioside A | 0.0045 | 0.005 | 0.003 | 0.01 | 0.0045 | 0.0025 | 0.001 |
| Citric acid | 0.15 | 0.15 | 0.06 | 0.15 | 0.15 | 0.15 | 0.15 |
| Phosphoric acid | — | — | 0.07 | — | — | — | — |
| Caramel | — | — | 0.14 | — | — | — | — |
| Caffeine | — | — | 0.01 | — | — | — | — |
| Citrus flavor | 0.1 | 0.05 | — | 0.1 | — | 0.1 | 0.1 |
| Lime flavor | — | 0.05 | — | — | 0.1 | — | — |
| “Cola-type” beverage | — | — | 0.05 | — | — | — | — |
| emulsion | | | | | | | |

| | |
|---|---|
| Water | Remainder up to 100 |

### Formulation Example 2

### Iced Tea

| Composition | 2.1 | 2.2 |
|---|---|---|
| Black tea extract | 1.4 | 1.4 |

| | |
|---|---|
| Water | Remainder up to 100 |

| | | |
|---|---|---|
| Flavor preparation (peach type) | 0.65 | 0.65 |
| Sugar | 3.5 | 3.0 |
| Citric acid (crystalline) | 1.2 | 1.2 |
| Ascorbic acid | 0.2 | 0.2 |
| Allulose | 3.5 | 4.0 |

### Formulation Example 3

### Instant Tea Drink

| Composition | 3.1 | 3.2 | 3.3 |
|---|---|---|---|
| Black tea, Ceylon, leaves | 94.00 | — | — |
| Green tea, China, leaves | — | 92.0 | — |
| Mate tea, Peru, leaves | — | — | 95.0 |
| Allulose | 6.0 | 8.0 | 5.0 |

### Formulation Example 4

### Instant Cappuccino Drink

| Composition | 4.1 | 4.2 |
|---|---|---|
| Coffee extract, spray-dried | 14.0 | 16.0 |
| Sugar | 25.0 | 20.0 |
| Allulose | 6.0 | 11.0 |
| Fat powder | 18.2 | 18.2 |

| | |
|---|---|
| Coffee creamer, foaming | Remainder up to 100 |

| | | |
|---|---|---|
| Hydrocolloids/emulsifiers | 1.8 | 1.8 |
| Lactose | 4.7 | 4.7 |

### Formulation Example 5

### Bitter Chocolate

| | | |
|---|---|---|
| Composition | 5.1 | 5.2 |

| | |
|---|---|
| Cocoa paste | Remainder up to 100 |

| | | |
|---|---|---|
| Cocoa butter | 11.70 | 11.70 |
| Sugar | 21.00 | 15.00 |
| Allulose | 9.00 | 15.00 |
| Whole milk | 3.00 | 3.00 |
| Lecithin | 0.2 | 0.2 |
| Vanillin | 0.035 | 0.035 |

### Formulation Example 6

### Fruit Gummies

| Composition | 6.1 | 6.2 |
|---|---|---|
| Allulose | 34.50 | 8.20 |
| Rebaudioside A | 0.003 | 0.003 |
| C* TruSweet 01750 iso syrup (Cerestar GmbH) | 1.50 | 2.10 |
| Gelatin 240 bloom | 8.20 | 9.40 |
| Polydextrose (Litesse ® Ultra, | — | 24.40 |
| Danisco Cultor GmbH) | | |
| Dye | 0.01 | 0.01 |
| Citrus flavor | 0.20 | — |
| Cherry flavor | — | 0.10 |

| | |
|---|---|
| Water | Remainder up to 100 |

### Formulation Example 7

### Sugar-Free Lozenges

| Composition | 7.1 | 7.2 | 7.3 |
|---|---|---|---|
| Water | 2.24 | 2.24 | 2.24 |
| Isomalt | Remainder up to 100 | | |
| Allulose | 2.40 | 2.60 | 3.0 |
| Sucralose | 0.03 | 0.03 | 0.03 |
| Acesulfame K | 0.050 | 0.050 | 0.050 |
| Citric acid | 0.050 | 0.050 | 0.050 |

### Formulation Example 8

### Toothpastes

| Composition | 8.1 | 8.2 | 8.3 |
|---|---|---|---|
| Gum base | Remainder up to 100 | | |
| Sorbitol, powdered | 40.00 | 40.00 | 40.00 |
| Isomalt, powdered | 9.50 | 9.50 | 9.50 |
| Xylitol | 2.00 | 1.00 | 1.00 |
| Mannitol D | 2.00 | 3.00 | 2.00 |
| Allulose | 1.00 | 1.00 | 1.00 |
| Acesulfame K | 0.10 | 0.10 | 0.10 |
| Emulgum/plasticizer | 0.30 | 0.30 | 0.30 |
| Sorbitol (70% water) | 13.00 | 13.00 | 13.00 |
| Glycerol | 1.00 | 1.00 | 1.00 |

### Formulation Example 9

### Toothpastes

| Composition | 9.1 | 9.2 | 9.3 |
|---|---|---|---|
| Deionized water | 36.39 | 36.39 | 36.39 |
| Glycerol | 20.00 | 20.00 | 20.00 |
| Solbrol M (sodium salt) | 0.15 | 0.15 | 0.15 |
| Sodium monofluorophosphate | 0.76 | 0.76 | 0.76 |
| Allulose | 0.20 | 0.25 | 0.30 |
| Dicalcium phosphate dihydrate | 36.00 | 36.00 | 36.00 |
| Aerosil 200 | 3.00 | 3.00 | 3.00 |
| Sodium carboxymethyl cellulose | 1.20 | 1.20 | 1.20 |
| Sodium lauryl sulfate | 1.30 | 1.30 | 1.30 |

### Formulation Example 10

### Mouthwash Concentrate

| Composition | 10.1 | 10.2 | 10.3 |
|---|---|---|---|
| Ethanol 96% | 42.00 | 42.00 | 42.00 |
| Cremophor RH 455 | 5.00 | 5.00 | 5.00 |
| Deionized water | 50.67 | 50.67 | 50.67 |
| Allantoin | 0.20 | 0.20 | 0.20 |
| Allulose | 0.10 | 0.20 | 0.30 |
| Color L-Blue 5000 (1% in water) | 0.03 | 0.03 | 0.03 |
| Flavor | 2.00 | 2.00 | 2.00 |

### Formulation Example 11

### Chewing Gum

| Composition | 11.1 | 11.2 |
|---|---|---|
| Chewing gum base, from Jagum T | 30.00 | 30.00 |
| Sorbitol, powdered | 39.00 | 39.00 |
| Isomalt ® (Palatinit GmbH) | 9.50 | 9.50 |
| Allulose | 2.00 | 2.00 |
| Mannitol | 3.00 | 3.00 |
| Aspartame ® | 0.10 | 0.10 |
| Acesulfame ® K | 0.10 | 0.10 |
| Emulgum ® (Colloides Naturels, Inc.) | 0.30 | 0.30 |
| Sorbitol, 70% | 14.00 | 14.00 |
| Glycerol | 1.00 | 1.00 |
| Mint flavor | 1.00 | — |
| Tutti frutti flavor | — | 2.00 |

### Formulation Example 12

### Gelatin Capsule

| | Composition | | |
|---|---|---|---|
| Gelatin shell: | 12.1 | 12.2 | 12.2 |
| Glycerol | 2.014 | 2.014 | 2.014 |
| Gelatin 240 bloom | 7.91 | 7.91 | 7.91 |
| Allulose | 0.065 | 0.085 | 0.10 |
| Allura Red | 0.006 | — | 0.011 |
| Brilliant blue | 0.005 | 0.011 | — |

### Core Composition:

| Vegetable oil triglyceride | Remainder up to 100 | | |
|---|---|---|---|
| (coconut oil fraction) | | | |
| Orange flavor | 10.0 | — | — |
| Peppermint flavor | — | 20.0 | — |
| Cherry flavor | — | — | 28.65 |
| Rebaudioside A 98% | 0.05 | 0.05 | — |
| 2-Hydroxypropyl menthyl carbonate | 0.33 | 0.20 | — |
| 2-Hydroxyethyl menthyl carbonate | — | 0.20 | 1.00 |
| (1R,3R,4S) Menthyl-3-carboxylic | — | 0.55 | — |
| acid-N-ethylamide (WS-3) | | | |
| (−)-Menthyl lactate (Frescolat ML) | — | 0.30 | — |
| Vanillin | 0.07 | — | 0.10 |

### Formulation Example 13

### Low-Fat Yogurt

| Composition | 13.1 | 13.2 | 13.3 | 13.4 |
|---|---|---|---|---|
| Allulose | 8.0 | 10.0 | 8.0 | 10.0 |
| Rebaudioside A | — | — | 0.025 | 0.025 |
| Saccharin | — | 0.3 | 0.3 | 0.3 |
| Tart cherry extract | 0.2 | 0.1 | 0.2 | 0.2 |

| | |
|---|---|
| Yogurt, 0.1% fat | Remainder up to 100 |

### Formulation Example 14

### Pudding

| | | | |
|---|---|---|---|
| Composition | 14.1 | 14.2 | 14.3 |

| | | | | | | |
|---|---|---|---|---|---|---|
| Cornstarch | 38 | g | 38 | g | 38 | g |
| Allulose | 38 | g | 28 | g | 19 | g |

| | | | | | |
|---|---|---|---|---|---|
| Sugar | — | 10 | g | 19 | g |
| Rebaudioside A | — | 0.05 | g | 0.08 | g |

| | | | | | | |
|---|---|---|---|---|---|---|
| Vanilla flavor (Symrise) | 0.2 | g | 0.2 | g | 0.2 | g |
| Quinoline yellow | 0.02 | g | 0.02 | g | 0.02 | g |
| Milk | 500 | ml | 500 | ml | 500 | ml |

### Formulation Example 15

### Brownies

| | | | |
|---|---|---|---|
| Composition | 15.1 | 15.2 | 15.3 |

| | | | | | | |
|---|---|---|---|---|---|---|
| Whole egg | 240.0 | g | 240.0 | g | 240.0 | g |
| Salt | 2.0 | g | 2.0 | g | 2.0 | g |

| | | | | | |
|---|---|---|---|---|---|
| Water | — | 40.0 | g | 60.0 | g |

| | | | | | | |
|---|---|---|---|---|---|---|
| Allulose, crystalline | 260.0 | g | 260.0 | g | 400.0 | g |

| | | | | |
|---|---|---|---|---|
| Allulose syrup, liquid | 200.0 | g | — | — |

| | | | | |
|---|---|---|---|---|
| Allulose syrup, solid | — | 160.0 | g | — |

| | | | | | | |
|---|---|---|---|---|---|---|
| Butter | 230.0 | g | 230.0 | g | 230.0 | g |
| Flour type 405 | 70.0 | g | 70.0 | g | 70.0 | g |
| Cocoa powder | 110.0 | g | 110.0 | g | 110.0 | g |
| Flavor (vanilla) | 5.0 | g | 5.0 | g | 5.0 | g |

1. Allulose in crystalline form, characterized in that it has a particle size distribution determined by the X_{c(min)} method, to which one, two, or all three of the following selection rules apply:

| Dispersion pressure | Minimum d10 | Minimum d50 | Minimum d90 |
|---|---|---|---|
| [kPa] | [μm] | [μm] | [μm] |
| 5 | >70 | >140 | >230 |
| 20 | >50 | >120 | >220 |
| 460 | >10 | >40 | >130 |

and/or
it has a particle size distribution determined by the X_{Fe(max)} method, to which one, two, or all three of the following selection rules apply:

| Dispersion pressure | Minimum d10 | Minimum d50 | Minimum d90 |
|---|---|---|---|
| [kPa] | [μm] | [μm] | [μm] |
| 5 | >100 | >220 | >340 |
| 20 | >70 | >200 | >330 |
| 460 | >8 | >65 | >170 |

and/or
it has a particle size distribution determined by the Xₐᵣₑₐ method, to which one, two, or all three of the following selection rules apply:

| Dispersion pressure | Minimum d10 | Minimum d50 | Minimum d90 |
|---|---|---|---|
| [kPa] | [μm] | [μm] | [μm] |
| 5 | >85 | >185 | >265 |
| 20 | >65 | >180 | >260 |
| 460 | >10 | >50 | >120 |

2. The allulose according to claim 1, characterized in that it has a b/l d50 value, to which the following applies:

| | Dispersion pressure | |
|---|---|---|
| Parameter | [kPa] | X_{c(min)} |
| b/l d50 | 5 | 0.52-1.0 |

and/or

| | | |
|---|---|---|
| b/l d50 | 20 | 0.53-1.0 |

and/or

| | | |
|---|---|---|
| b/l d50 | 460 | 0.60-1.0 |

and/or

| | Dispersion pressure | |
|---|---|---|
| Parameter | [kPa] | X_{Fe(max)} |
| b/l d50 | 5 | 0.52-1.0 |

and/or

| | | |
|---|---|---|
| b/l d50 | 20 | 0.55-1.0 |

and/or

| | | |
|---|---|---|
| b/l d50 | 460 | 0.65-1.0 |

and/or

| | Dispersion pressure | |
|---|---|---|
| Parameter | [kPa] | Xₐᵣₑₐ |
| b/l d50 | 5 | 0.52-1.0 |

and/or

| | | |
|---|---|---|
| b/l d50 | 20 | 0.55-1.0 |

and/or

| | | |
|---|---|---|
| b/l d50 | 460 | 0.65-1.0 |

3. A process for the production of the crystalline allulose of claim 1, comprising or consisting of the following steps:
(a) providing an aqueous allulose preparation, preferably an evaporator concentrate (“allulose syrup”), comprising or consisting of the following components, based on the dry matter contained in the preparation:
(i) about 90 to about 99% by weight allulose;
(ii) about 0.5 to about 10% by weight fructose;
(iii) 0 to about 5% by weight carbohydrates other than allulose and fructose;
with the proviso that the quantities add up to 100% by weight;
(b) optionally treating the allulose preparation of step (a) with a decolorizing agent;
(c) concentrating the preparation from step (a) or step (b) until the saturation concentration is exceeded;
(c) adding allulose seed crystals to the supersaturated preparation of step (c); and
(d) cooling the preparation of step (c) at a rate of from 0.1 to about 1 K/h until a crystal content from about 30 to about 50% by weight is reached; and
(e) separating and optionally drying the crystals.

4. The process according to claim 3, characterized in that the preparation of step (a) has an allulose content ≥90% by weight, preferably ≥91% by weight, preferably ≥92% by weight, preferably ≥93% by weight, preferably ≥94% by weight, preferably ≥95% by weight, preferably ≥96% by weight, preferably ≥97% by weight, preferably ≥98% by weight, based on the dry matter contained in the preparation in each case.

5. The process according to claim 3, characterized in that the preparation of step (a) has an allulose content ≤99% by weight, preferably ≤98% by weight, preferably ≤97% by weight, preferably ≤96% by weight, preferably ≤95% by weight, preferably ≤94% by weight, preferably ≤93% by weight, preferably ≤92% by weight, preferably ≤91% by weight, based on the dry matter contained in the preparation in each case.

6. The process according to claim 3, characterized in that the preparation of step (a) has a fructose content from about 1 to about 3% by weight fructose, based on the dry matter contained in the preparation.

7. The process according to claim 6, characterized in that the preparation of step (a) has a fructose content from about 1.5 to about 2.5% by weight fructose, based on the dry matter contained in the preparation.

8. The process according to claim 3, characterized in that the preparation of step (a) has a fructose content from about 0.5 to about 1% by weight fructose, or from about 1 to about 1.5% by weight, or from about 1.5 to about 2.0% by weight, or from about 2.0 to about 2.5% by weight, or from about 2.5 to about 3.0% by weight, or from about 3.0 to about 3.5% by weight, or from about 3.5 to about 4.0% by weight, or from about 4.0 to about 4.5% by weight, or from about 4.5 to about 5.0% by weight, or from about 5.0 to about 5.5% by weight, or from about 5.5 to about 6.0% by weight, or from about 6.0 to about 6.5% by weight, or from about 6.5 to about 7.0% by weight, or from about 7.0 to about 7.5% by weight, or from about 7.5 to about 8.0% by weight, or from about 8.0 to about 8.5% by weight, or from about 8.5 to about 9.0% by weight, or from about 9.5 to about 10% by weight, based on the dry matter contained in the preparation in each case.

9. The process according to claim 3, wherein preparations are used in step (a) which have a dry matter content from about 55 to about 98% by weight.

10. The process according to claim 9, characterized in that preparations are used in step (a) which have a dry matter content from about 75 to about 95% by weight; optionally wherein preparations are used in step (a) which have a solids content from about 75 to about 80% by weight, or from about 80 to about 85% by weight, from about 85 to about 90% by weight, or from about 90 to about 95% by weight.

11. (canceled)

12. The process of claim 3, wherein the concentration of step (c) is carried out at a temperature from about 50 to about 75° C.

13. The process of claim 3, wherein seed crystals are added to the supersaturated preparation in amounts of about 1 to about 3% by weight, based on the preparation.

14. An oral preparation comprising the crystalline allulose of claim 1.

15. The oral preparation of claim 14, wherein the oral preparation is selected from the group consisting of a food, an oral and dental care agent, and a pharmaceutical preparation.

16. The oral preparation of claim 15, wherein the oral preparation is the food and is selected from the group consisting of a beverage, a baked good, a grain product, a snack, a confectionery, a dairy product, a fruit preparation, a seasoning, a vegetable preparation, a meat product, a spice and a spice mix.

17. The oral preparation of claim 16, wherein
the oral preparation is a beverage selected from the group consisting of coffee, tea, iced tea, wine, wine-based drinks, beer, beer-based drinks, liqueurs, schnapps, brandy, (carbonated) fruit-based lemonades, (carbonated) isotonic drinks, flavored mineral water, (carbonated) soft drinks, nectars, spritzers, fruit and vegetable juices, fruit or vegetable juice preparations, instant cocoa drinks, instant tea drinks, instant coffee drinks, and instant fruit drinks; or
the oral preparation is a baked good, grain product or snack selected from the group consisting of bread, dry cookies, cakes, muffins, donuts, other pastries, baked or fried potato chips or potato dough products, extrudates based on corn or peanuts, breakfast cereals, granola bars, and precooked ready-made rice products; or
the oral preparation is a confectionery or fruit preparation selected from the group consisting of chocolates, chocolate bar products, other bar products, fruit gummies, hard and soft caramels, chewing gum, tablets, mints, coatings (fondants), jams, sherbets, fruit sauces, and fruit fillings; or
the oral preparation is a milk product selected from the group consisting of milk drinks, buttermilk drinks, milk-based ice cream, yogurt, kefir, cream cheese, soft cheese, hard cheese, dried milk powder, whey, whey drinks, butter, buttermilk, partially or completely hydrolyzed products containing milk protein, products made from soy protein or other soybean fractions, fruit drinks with soy protein, preparations containing soy lecithin, fermented products such as tofu or tempeh or products made therefrom), and products made from other plant-based protein sources; or
the oral preparation is a seasoning or vegetable preparation selected from the group consisting of ketchup, sauces, dried vegetables, frozen vegetables, pre-cooked vegetables, cooked vegetables, mayonnaise, tartar sauce, dressings, ready-made meals, dry soups, instant soups, pre-cooked soups, spices, spice mixes, and seasonings; or
the oral preparation is a meat product selected form the group consisting of ham, fresh sausage, or raw sausage preparations, and seasoned or marinated fresh or cured meat products; or
the oral preparation is a spices or spice mix are selected from the group consisting of sweetener enhancers, flavor enhancers, and masking agents.

18. (canceled)

19. (canceled)

20. (canceled)

21. (canceled)

22. (canceled)

23. (canceled)

24. The oral preparation of claim 15, wherein the oral preparation is the oral and dental care agent and is selected from the group consisting of toothpastes, mouthwashes, and chewing gum.

25. The oral preparation of claim 15, wherein the oral preparation is the pharmaceutical agent and is selected from the group consisting of solid, tableted, or coated active ingredients, active ingredient mixtures, and liquid products.

26. A method comprising a step of:
covering up and/or masking an unpleasant, astringent, and/or metallic aftertaste of an active pharmaceutical ingredient with the allulose of claim 1; or
covering up and/or masking the unpleasant taste notes (astringent, metallic, tarry, greasy) of a flavor enhancer with the allulose of claim 1; or
covering up and/or masking an unpleasant taste notes (astringent, metallic, tarry, greasy) of a flavoring with the allulose of claim 1.

27. A pharmaceutical comprising the allulose of claim 1, and one or more active pharmaceutical ingredients including a non-steroidal anti-inflammatory drug (NSAID);
wherein the allulose covers up and/or masks an unpleasant, astringent, and/or metallic aftertaste of one of the active pharmaceutical ingredients:
optionally wherein the one or more active pharmaceutical ingredients includes ASPIRIN (acetylsalicylic acid), MINOXIDIL (6-piperidin-1-ylpyrimidine-2,4-diamine-3-oxide), ERYTHROMICIN, DIMETINDENE (FENISTIL) (RS-dimethyl(2-(3-[pyridin-2-yl)ethyl]-1H-inden-2-yl)ethyl)amine), BETAMETHOSONE (8S,9R,10S,11S,13S,14S,16S,17R)-9-fluoro-11,17-(2-hydroxyacetyI)-10,13,16-trimethyl-6,7,8,9,10,11,12,13,14,15,16,17-dodecahydro-3H-cyclopenta(alpha)-phenanthrene-3-one, IBUPROFEN (RS)-2-(4-(2-methylpropyl)phenyl)propanoic acid), KETOPROFEN (RS)2-(3-benzoylphenyl)propionic acid), DICLOFENAC, Metronidazole (2-(2-methyl-5-nitro-1H-imidazol-1-yl)ethanol, ACYCLOVIR (2-amino-1,9-dihydro-9-((2-hydroxyethoxy)methyl)-6H-purin-6-one), IMIQUIMOD (3-(2-methylpropyl)-3,5,8-triazatricyclo[7.4.0.0.2,6]trideca-1(9),2(6),4,7,10,12-hexaen-7-amine, TERBINAFIN [(2E)-6,6-dimethylhept-2-en-4-yn-1-yl](methyl)(naphthalen-1-ylmethyl)amine), CICLOPYRAXOLAMINE (6-cyclohexyl-1-hydroxy-4-methylpyridin-2(1H)-one), or a mixture thereof.

28. (canceled)

29. (canceled)

30. (canceled)
